(19) United States
(12) Patent Application Publication
(10) Pub. No.: US 2021/0252132 A1
(43) Pub. Date: Aug. 19, 2021

(54) COMPOSITION AND METHOD FOR STABILISING VACCINES IN A SOLID DOSAGE FORMAT

(71) Applicant: University College Cork-National University of Ireland, Cork, Cork (IE)

(72) Inventors: Agnese Donadei, Cork (IE); Olivia Flynn, Cork (IE); Anne Moore, Cork (IE)

(21) Appl. No.: 17/251,965

(22) PCT Filed: Jun. 12, 2019
(86) PCT No.: PCT/EP2019/065392

§ 371 Date: Dec. 14, 2020

(30) Foreign Application Priority Data
Jun. 14, 2018 (EP) 18177686.5

(51) Int. Cl. A61K 39/13; A61K 39/235

(52) U.S. Cl. CPC A61K 39/13; A61K 2039/55583; A61K 39/235

(57) ABSTRACT

A composition for stabilising a vaccine in a solid dosage format is provided wherein the composition comprises an antioxidant, such as glutathione, a monosaccharide or disaccharide sugar, such as trehalose, a polyol sugar, such as sorbitol, one or more salts, such as magnesium chloride and sodium glutamate, and a vaccine. The composition may also comprise an aqueous soluble polymer, such as polyvinyl alcohol (PVA). A preferred composition comprises 40 mM glutathione, 20% w/v trehalose, 3% w/v sorbitol, 5% w/v PVA, 3% w/v magnesium chloride and 3% w/v sodium glutamate. Also provided is a method of stabilising a vaccine in a solid dosage format, the method comprising drying the stabilising composition to provide the vaccine in the solid dosage format. The composition and method may be used to stabilise any suitable vaccine, such as poliovirus or adenovirus, in a solid dosage format, such as microneedle patches or wafers.

### FIELD OF THE INVENTION

The present invention relates to a composition and method for stabilising vaccines in a solid dosage format.

### BACKGROUND OF THE INVENTION

Vaccination is the most effective method of preventing infectious diseases. Widespread immunity due to vaccination is largely responsible for the worldwide eradication of smallpox and the restriction of diseases such as polio, measles, and tetanus from much of the world. The World Health Organization (WHO) reports that licensed vaccines are currently available for twenty-six different preventable infections. Vaccines are often delivered by injection requiring administration by a trained health worker and use of sterile needles and syringes. This results in hazardous waste as disposal of used needles, syringes and glass vials is required. Furthermore, vaccines are labile biologics. Vaccines in liquid dosage format require costly cold chain storage and distribution and reconstitution in some cases. These logistic costs are important economic limiting factors. Given the cost of cold chain storage, vaccine manufacturers are reluctant to over-produce vaccines that may be surplus to requirements and are de-risking the costs and resource implication of cold chain storage of vaccines. A solution is needed to the problems of (i) increased cost and complexity of injection-based immunization with vaccines such as IPV and (ii) expensive cold chain storage and distribution which makes stockpiling vaccines, such as polio vaccines, difficult.

An RNA virus is a virus that has RNA (ribonucleic acid) as its genetic material. The poliovirus is a non-enveloped RNA virus with a protein capsid that encapsulates the ribonucleic acid. The polio vaccine is used to immunise against poliomyelitis (polio), which is an infectious disease caused by poliovirus strains type 1, type 2 and type 3. The polio vaccine is part of national routine immunization schedules and polio immunization campaigns have almost globally eradicated these three strains. Two vaccine strategies have been used to generate this successful outcome. Oral polio vaccines (OPV), developed by Sabin, are administered by drops into the mouth and are relatively inexpensive to purchase and administer. However, this is a live attenuated vaccine and on rare occasions can cause vaccine-associated paralytic poliomyelitis. Inactivated (killed) polio vaccines (IPV), developed by Salk, involve the production of infectious poliovirus, which is then inactivated and formulated for systemic administration with a needle-and-syringe. IPV is currently given by intramuscular injection. As such, it needs to be administered by a trained health worker and sterile needles/syringes are required. IPV is consequently related to hazardous waste requiring disposal of used needles, syringes and glass vials. Although IPV is one of the safest vaccines in use, there are increased bio-safety concerns at the point of manufacture. This is largely responsible for the higher cost of this vaccine. As part of the WHO's Global Polio Eradication and Endgame Strategic Plan, an action plan is being implemented to replace OPV with IPV and to use safer Sabin strains to manufacture inactivated polio vaccine, termed Sabin IPV (sIPV). The switch from OPV to IPV will require significant increases in cost and complexity of immunization, as vaccinators will need to administer the vaccine by injection rather than oral drops. Moreover, due to planned changes in the composition of polio vaccines from OPV to IPV and from trivalent to bivalent vaccines, and given the constraints of cold chain storage, there is now a global shortage of polio vaccines for routine immunization and for stockpiling in case of an outbreak.

A DNA virus is a virus that has DNA as its genetic material and replicates using a DNA-dependent DNA polymerase. Notable diseases like smallpox, herpes, and chickenpox are caused by DNA viruses. Attenuated recombinant viral vectors are a powerful technology for delivering antigens from a wide range of infectious diseases and tumours. The capacity to infect cells and express encoded antigens that may be secreted or presented to T cells ensures highly efficient induction of both humoral and cytotoxic immune responses. This provides a key advantage over subunit vaccines. Viral vectors also have intrinsic adjuvant properties, as they possess pathogen-associated molecular patterns which activate innate immunity. Recombinant vaccine vectors that are demonstrating promise include viruses in the RNA Rhabdoviridae family (for example recombinant Vesicular Stomatitis Virus, rVSV) and DNA viruses in the Poxviridae family, particularly orthopoxviruses and avipoxviruses and within the Adenoviridae family. Adenoviruses are non-enveloped DNA viruses that typically cause respiratory illnesses, such as a common cold, conjunctivitis, croup, bronchitis and pneumonia. Genetically attenuated, recombinant adenoviruses of human and simian origin are demonstrating high potential across several infectious diseases of global importance. Adenoviral vectored vaccines can induce potent antigen-specific B- and T-cell immune responses to the antigen(s) of interest. There remains an unmet need to develop cold chain-free technologies to ensure effective delivery to all geographical regions, including resource-poor settings.

Previous efforts to stabilise the polio vaccine have focused on stabilising the polio vaccine in a liquid form or by lyophilising the vaccine (sIPV) to create a solid form. This freeze-drying method relies on complex freeze-dry cycles and defined parameters to form a vaccine “cake”. Furthermore, specialist lyophilisation equipment is required and the lyophilised cake must be resuspended for injection.

### SUMMARY OF THE INVENTION

According to a first aspect of the present invention, there is provided a composition for stabilising a vaccine in a solid dosage format, wherein the composition comprises, consists essentially of or consists of:
an antioxidant;
a monosaccharide or disaccharide sugar;
a polyol sugar;
one or more salts;
a vaccine; and
optionally an aqueous soluble polymer,
   wherein the composition is formulated such that at least 45% of the initial potency of the vaccine is retained subsequent to stabilising the vaccine in the solid dosage format.

According to a second aspect, there is provided a composition for stabilising a vaccine in a solid dosage format, the composition comprising, consisting essentially of or consisting of:
20 to 80 mM antioxidant;
10 to 40% w/v monosaccharide or disaccharide sugar;
3-8% w/v polyol sugar;
0.5-8% w/v of each of one or more salts;
0-5% w/v aqueous soluble polymer; and
a vaccine.

According to a third aspect of the present invention, there is provided a vaccine in a solid dosage format comprising, consisting essentially of, or consisting of a composition according to a first or second aspect of the present invention, wherein the composition has been dried to provide the vaccine in the solid dosage format.

According to a fourth aspect of the present invention, there is provided a method of stabilising a vaccine in a solid dosage format, the method comprising the steps of:
providing a composition according to a first or second aspect of the present invention; and
drying the composition to provide the vaccine in the solid dosage format.

According to a fifth aspect of the present invention, there is provided use of a composition according to a first or second aspect of the present invention to stabilise a vaccine in a solid dosage format.

### BRIEF DESCRIPTION OF THE FIGURES

The invention will be more clearly understood from the following description of some embodiments thereof, given by way of example only, with reference to the following figures in which:

FIG. 1 shows particle size for IPV type 3 after a buffer change process in saline solution and stored at +4° C. (“fresh liquid”—positive control).

FIG. 2 shows particle size for IPV type 3 after a buffer change process in saline solution, subsequently formulated with water and exposed at +20° C./10 mBar for 24 hours drying (negative control).

FIG. 3 shows particle size for IPV type 3 after a buffer change process in saline solution, formulated with glutathione 20 mM and exposed at +20° C./10 mBar for 24 hours drying.

FIG. 4 shows particle size for IPV type 3 after a buffer change process in saline solution, formulated with glutathione 20 mM, sorbitol 10% w/v, trehalose 15% w/v and exposed at +20° C./10 mBar for 24 hours drying.

FIG. 5 shows the native conformation of IPV type 3 (Salk) after a buffer change process in saline solution, formulated with glutathione 20 mM and exposed at +20° C./10 mBar for 24 hours drying. It was analysed for its intrinsic fluorescence.

### DETAILED DESCRIPTION OF THE INVENTION

The present inventors have developed a composition and method for stabilising vaccines that can be used to provide vaccines in a solid dosage format without substantial loss of vaccine potency. The provision of a vaccine in a solid dosage format is advantageous as providing a non-injection based vaccine dosage format overcomes the problems inherent in injection-based immunization. The provision of solid dosage formats, such as patches for buccal, sublingual, skin administration or administration to other bodily surfaces, eliminates the requirement for vaccines to be injected. This allows for alternative routes of administration for vaccination, such as polio vaccination. The invention thus opens up feasibility to administer a vaccine by the oral, intradermal and mucosal routes using capsules, microneedles, films and wafers. Oral vaccines have been shown to be effective when vaccinations were administered by volunteer staff without formal training and there is no risk of blood contamination. Microneedles use pointed projections fabricated into arrays that can create vaccine delivery pathways through the skin. Administration using these vaccine delivery systems and by these routes potentially increases the effectiveness of vaccination, while requiring less vaccine than injection.

Furthermore, vaccines in solid dosage format commonly are more stable and less prone to damage or to spoilage by thermal stresses in transport and storage. Such stability reduces the need for cold chain storage and distribution which is often required in order to keep vaccines within a restricted temperature range from the manufacturing stage to the point of administration. This, in turn, may decrease costs of vaccines. The composition and method of the present invention both chemically and physically stabilise vaccines, particularly virus vaccines, including whole inactivated and live vaccines, in solid dosage formats such that the vaccines can be stored, handled and used at room temperature. Thermostabilising the vaccine so that cold chain logistics are eliminated allows vaccine stockpiling in regular drug distribution systems. This would have a significant impact on solving vaccine storage and distribution. The use of a solid dosage format for vaccination thus allows an effective vaccination in the absence of refrigerated storage conditions, reducing costs and increasing vaccine stability in unfavourable environmental conditions. For example, in countries where cold chain is a significant issue in vaccine distribution, improved stability would assist in an increased availability of vaccine stocks for potential disease outbreaks and more effective vaccination.

The antioxidant may comprise one or more amino acids, such as sulphur-containing amino acids. The antioxidant may be selected from the group consisting of cysteine, methionine, tryptophan, taurine, glutathione, glycine, glutamic acid, lipoic acid, N-acetylcysteine, ascorbic acid (vitamin C) and combinations thereof. In certain embodiments, the antioxidant may be selected from the group consisting of glutathione, vitamin C, cysteine, glycine, glutamic acid and a combination comprising cysteine (Cys), glutamic acid (Glu) and glycine (Gly). The antioxidant may be glutathione. The antioxidant (e.g. glutathione, vitamin C, cysteine, glycine, glutamic acid or a combination comprising cysteine (Cys), glutamic acid (Glu) and glycine (Gly)) may be present within the composition at a concentration of 20 to 80, 30 to 50 or 25 to 35 mM. The antioxidant may be present within the composition at a concentration of 20, 25, 30, 35, 40, 45, 50, 55, 60, 65, 70, 75 or 80 mM. The antioxidant (e.g. glutathione, vitamin C, cysteine, glycine, glutamic acid or a combination comprising cysteine (Cys), glutamic acid (Glu) and glycine (Gly)) may be present within the composition at a concentration of 20-40 mM, for example when the vaccine is for poliovirus. The antioxidant (e.g. glutathione) may be present within the composition at a concentration of 40-80 mM, for example when an adenovirus vaccine is used. The antioxidant (e.g. glutathione) may be present within the composition at a concentration of 40 mM. The inventors have shown that vitamin C may be used in place of glutathione. The inventors have also shown that cysteine, glycine, glutamic acid or a combination comprising all three may be used in place of glutathione. Without being bound by theory, it is hypothesized that smaller anti-oxidant molecules could be more potent than larger anti-oxidant molecules, such as glutathione, when higher amounts of polymers are used, due to the capacity of the smaller molecules to permeate throughout the polymer-based formulation, whereas larger antioxidants may be sterically excluded in all spaces by the polymer. In certain embodiments therefore, when an aqueous soluble polymer is present, the antioxidant may comprise a smaller molecule.

The monosaccharide or disaccharide sugar may be any suitable monosaccharide or disaccharide sugar. The monosaccharide sugar may be selected from glucose or galactose. The disaccharide sugar may be selected from the group consisting of trehalose, sucrose, lactose, maltose and combinations thereof. The disaccharide sugar may be trehalose. The monosaccharide or disaccharide sugar (e.g. trehalose) may be present within the composition at a concentration of 10 to 40% w/v, 10 to 25% w/v, 10 to 20% w/v, 15 to 25% w/v, 15 to 30% w/v or 15 to 20% w/v. The monosaccharide or disaccharide sugar (e.g. trehalose) may be present within the composition at a concentration of 10%, 15%, 20%, 25%, 30%, 35% or 40% w/v. The monosaccharide or disaccharide sugar (e.g. trehalose) may be present within the composition at a concentration of 15% w/v or 20% w/v. The monosaccharide or disaccharide sugar (e.g. trehalose) may be present within the composition at a concentration of 15% to 25% w/v, for example where the vaccine is for poliovirus, or at a concentration of 20% to 30% w/v, for example when an adenovirus-based vaccine is used. The monosaccharide or disaccharide sugar (e.g. trehalose) may be present within the composition at a concentration of 15% to 30% w/v. The monosaccharide or disaccharide sugar (e.g. trehalose) may be present within the composition at a concentration of 20% w/v.

The polyol sugar may be selected from the group consisting of sorbitol, mannitol, maltitol, lactitol, xylitol, iosmalt, erythritol and combinations thereof. The polyol sugar may be sorbitol. The polyol sugar has a stabilising effect on the vaccine. The polyol sugar (e.g. sorbitol) may be present within the composition at a concentration of 3-8% w/v, 3-7% w/v, 3-6% w/v or 3-5% w/v. The polyol sugar (e.g. sorbitol) may be present at a concentration of 3% w/v, 4% w/v, 5% w/v, 6% w/v, 7% w/v or 8% w/v. The polyol sugar (e.g. sorbitol) may be present at a concentration of 3% w/v or 5% w/v. The polyol sugar (e.g. sorbitol) may be present within the composition at a concentration 3-5% w/v. The polyol sugar (e.g. sorbitol) may be present at a concentration of 3% w/v. The inventors have shown that a polyol sugar (e.g. sorbitol) is required for stability of IPV.

The composition may comprise an aqueous soluble polymer. The aqueous soluble polymer may be a commercially available aqueous soluble polymer. For example, it may be selected from the group consisting of polyvinyl alcohol (PVA), carboxymethyl cellulose (CMC), polyvinyl pyrrolidone (PVP), hydroxypropyl methylcellulose (HPMC), ethylcellulose, polyacrylamide, polyacrylic acids, polyacrylates, acrylic/maleic copolymers, and combinations thereof. Typically, the aqueous soluble polymer may be PVA. The aqueous soluble polymer provides the composition with mechanical strength, in particular, where the vaccine is being provided in dissolving microneedle patches. The aqueous soluble polymer (e.g. PVA) may be present within the composition at a concentration of 0% to 6% weight/volume (w/v), 0% to 5.5% w/v or 0% to 5% w/v. The concentration may be 5% w/v or less, 4% w/v or less, or 3.5% w/v or less, or 3% w/v or less. The inventors have found that an optimal PVA concentration is 3% w/v or less. Accordingly, the aqueous soluble polymer (e.g. PVA) may be present within the composition at a concentration of 3% w/v or less, 2.5% w/v or less, 2% w/v or less, 1.5% w/v or less, 1% w/v or less or 0.5% w/v or less. The concentration of the aqueous soluble polymer (e.g. PVA) may be 1.5% w/v. The concentration of the aqueous soluble polymer (e.g. PVA) may be 0.5% to 3% w/v, for example where the vaccine is for adenovirus. The concentration of the aqueous soluble polymer (e.g. PVA) may be 1.5% to 5% w/v, for example where the vaccine is for poliovirus. The concentration of the aqueous soluble polymer (e.g. PVA) may be 0.5% to 5% w/v.

The composition comprises one or more salts, such as magnesium chloride, sodium glutamate or a combination thereof. The salt has a stabilising effect on the vaccine. The one or more salts (e.g. magnesium chloride, sodium glutamate or a combination thereof) may each be present within the composition at a concentration of 0.5-8% w/v, 1-7% w/v, 1-6% w/v, 1-5% w/v, 2-4% w/v, 2.5-3.5% w/v or 3% w/v. The composition may comprise magnesium chloride at 3% w/v and/or sodium glutamate at 3% w/v. The composition may comprise both magnesium chloride and sodium glutamate each at 2.5% to 3.5% w/v, preferably 3% w/v. The composition may comprise one or more salts (e.g. magnesium chloride and/or sodium glutamate) each at 0.5 to 3% w/v, for example, when it comprises adenovirus. The composition may comprise one or more salts (e.g. magnesium chloride and/or sodium glutamate) each at 3% w/v, for example, where the vaccine is for poliovirus.

In certain embodiments, the composition includes one or more of the following: a) a combination of glutathione and sorbitol;
b) a combination of glutathione and trehalose;
c) a combination of glutathione and salt;
d) a combination of glutathione and poly-vinyl alcohol (PVA);
e) a combination of sorbitol and trehalose;
f) a combination of sorbitol and salt;
g) a combination of sorbitol and poly-vinyl alcohol; and
h) a combination of all of the above.

In certain embodiments, the composition comprises, or consists essentially of:
glutathione, vitamin C, glycine, cysteine, glutamic acid or a combination of glycine, cysteine and glutamic acid as the antioxidant;
trehalose as the disaccharide sugar;
sorbitol as the polyol sugar; and
magnesium chloride and/or sodium glutamate as the one or more salts.

In certain embodiments, the composition further includes PVA as the aqueous soluble polymer.

In certain embodiments, the composition comprises, consists essentially of, or consists of:
antioxidant (e.g. glutathione, vitamin C, glycine, cysteine, glutamic acid or a combination of glycine, cysteine and glutamic acid) at a concentration of 20 to 80 mM, optionally 40 mM;
monosaccharide or disaccharide sugar (e.g. trehalose) at a concentration of 10 to 40% w/v, optionally 20% w/v;
polyol sugar (e.g. sorbitol) at a concentration of 3-8% w/v, optionally 3% w/v;
an aqueous soluble polymer (e.g. PVA) at a concentration of 0-5% w/v, optionally 3% w/v or less (e.g. 1.5% w/v); and
one or more salts (e.g. magnesium chloride and/or sodium glutamate) each at a concentration of 0.5-8% w/v, optionally 0.5-5% w/v, optionally 3% w/v.

In certain embodiments, the composition comprises, consists essentially of, or consists of:

40 mM glutathione;
20% w/v trehalose;
3% w/v sorbitol;
1.5% w/v PVA;
3% w/v magnesium chloride; and
3% w/v sodium glutamate.

The composition may also include a buffer. A buffer may be used to provide the required pH if necessary, for example for stability of the aqueous soluble polymer or the antioxidant. For example, PVA is stable at a pH of between 6 and 7.4.

Typically, the composition does not include urea. Typically, the composition does not include cyclodextrins.

The composition may be used to provide stability for monovalent and multivalent vaccines in a solid dosage format. The vaccine may comprise any suitable type of virus vaccine which is comprised of an RNA or DNA nucleic acid and a coat. The coat is comprised of capsid proteins and may have an additional lipid and protein single or double envelope layer on the outside. In certain embodiments, the vaccine comprises an RNA virus vaccine, e.g. a non-enveloped RNA virus vaccine. RNA viruses may include picornaviruses, influenza, rotaviruses, alphaviruses, arboviruses, filoviruses, morbilliviruses, hepatitis viruses, alphaviruses such as Vesicular Stomatitis Virus (rVSV), etc. The vaccine may be for polio or for other picornaviruses, such as foot and mouth disease virus (FMDV) and enteroviruses, or mixtures of these. In certain embodiments, the vaccine comprises a DNA virus vaccine, e.g. a live DNA virus, a non-enveloped DNA virus or a live DNA non-enveloped virus. In certain embodiments, the virus may be an adenovirus. The vaccine may be a live attenuated vaccine, for example DNA viruses including recombinant adenoviruses, poxviruses, vaccinia virus or RNA viruses such as alphaviruses such as Vesicular Stomatitis Virus (rVSV).

Adenovirus, includes but is not limited to, human Ad5, Ad2, Ad6, Ad24 serotype, chimpanzee, sheep or other adenoviruses, particularly recombinant adenoviral virus for use in prophylactic or therapeutic vaccines for humans or veterinary species. The vaccine may comprise an inactivated virus vaccine, such as inactivated polio vaccine, FMDV or whole inactivated influenza vaccine, whole virus vaccines, whole inactivated virus vaccines, recombinant vaccines, and vector vaccines, virus-like particle (VLP) vaccines. In particular, the vaccine may comprise an inactivated virus, e.g. an inactivated whole virus vaccine. The virus may be any virus suitable for use in a vaccine. The virus may be a non-enveloped virus, an enveloped virus, a recombinant virus or a combination thereof. The virus may be a picornavirus. The picornavirus may be selected from the group consisting of enterovirus, aphthovirus, cardiovirus, rhinovirus and hepatovirus genera. In particular, the picornavirus may be an enterovirus. Typically, the enterovirus may be poliovirus. The apthovirus may cause foot and mouth disease. Where the virus is poliovirus, the poliovirus may be selected from Salk IPV (conventional IPV) and Sabin oral (OPV) or inactivated poliovirus vaccine (sIPV). The poliovirus may be type 1, 2 or 3, for example, Salk type 1, 2 or 3 or Sabin type 1, 2 or 3.

The composition may comprise one or more adjuvants, stabilizers or preservatives. Adjuvants enhance the immune response of the antigen, stabilizers increase the storage life, and preservatives allow the use of multidose vials. In particular, the composition may comprise one or more excipients selected from the group consisting of aluminium salts or gels, antibiotics, formaldehyde, monosodium glutamate and 2-phenoxyethanol.

The composition comprising the vaccine in liquid format may be dried to provide the vaccine in a solid dosage format. The composition may be dried at ambient temperature or any temperature suitable for use with thermosensitive biological materials. Lyophilisation is not required for drying. The solid dosage format may be selected from the group consisting of microneedles, capsules, wafers, films, microneedle patches (e.g. dissolving microneedle patches) and patches, such as patches for buccal, sublingual, skin, vaginal or anal administration. Typically, the solid dosage format is not a foam. The vaccine in solid dosage format may be administered by any suitable means of administration, such as, by oral, transcutaneous or intradermal administration. In certain embodiments, the vaccine in solid dosage format is administered orally. In certain embodiments, the vaccine in solid dosage format is administered by transcutaneous administration. As such, the requirement for reconstitution to a liquid and administration by injection is eliminated.

In the method of stabilising a vaccine in a solid dosage format, providing a composition may comprise preparing the composition for stabilising a vaccine in a solid dosage format by combining excipients as described herein and a vaccine. The vaccine may be any suitable vaccine, for example, existing polio vaccines, adenovirus vaccines and other types of vaccines.

Drying may comprise using a vacuum. This accelerates drying. The method does not require lyophilisation. The composition is dried until only a small amount of residual moisture remains in the composition. The appropriate or preferred amount of residual moisture depends on the type of vaccine. This may be determined by those of skill in the art by consulting appropriate literature. In certain cases, the composition (e.g. a vaccine against poliovirus) is dried until the residual moisture is 3% or less or 1% or less (J. C. May, et al. Measurement of final container residual moisture in freeze-dried biological products (Dev. Biol. Stand., 74 (1992), pp. 153-164)). The drying time is selected depending on the vaccine type in order to maximize the vaccine recovery in the claimed composition. The appropriate drying time for a particular vaccine may be determined by those of skill in the art. The vaccine (e.g. a vaccine against poliovirus) may be dried for a time period ranging from 24 hours to 48 hours, typically 30 or 36 hours. Typically, a minimum drying time of 24 hours is required. The method of the invention merely relies on drying at suitable (e.g. ambient) temperature or by vacuum to accelerate drying. Crucially, it can be independent of lyophilisation.

The composition and method of the invention can be used to produce a solid dosage format that stabilises viruses, such as inactivated polio virus vaccine (IPV) and live adenovirus vaccines. The stabilising composition and method can be used for monovalent, bivalent and trivalent vaccines. The stabilising composition and method can be used with both sIPV and conventional IPV and can be used to prevent poliomyelitis caused by poliovirus strains type 1, type 2 and type 3.

The present invention thus addresses two problems in the vaccine field, in particular, the polio field, firstly the issue of costs and logistics surrounding injection-based immunisation and secondly the issue of vaccine stability and cold storage.

The vaccine in solid dosage format may be administered to a subject in need thereof, e.g. a subject who is at risk of being infected by the virus in question. The vaccine in solid dosage format may be administered to the subject via any suitable route. In particular, the vaccine in solid dosage format may be administered orally, by transcutaneous routes, by mucosal routes or topically. The vaccine may be administered via microneedles, patches, films, wafers or any other suitable solid dosage format. The vaccine in solid dosage format may be administered by a non-medically trained person. The vaccine in solid dosage format may be stored and transported at room temperature. Advantageously, the vaccine may be administered without being reconstituted into a liquid. The solid dosage format may be a microneedle. The solid dosage format may be a wafer.

The composition is formulated such that at least 30%, 35%, 40%, 45% or 50% of the initial potency of the vaccine is retained subsequent to stabilising the vaccine in the solid dosage format. Typically, at least 55%, 60%, 65%, 70%, 75%, 80%, 85%, 90%, 95%, 96%, 97%, 98% or 99% of initial vaccine potency is retained. The initial potency of the vaccine refers to the potency of the vaccine prior to stabilising the vaccine in the solid dosage format, e.g. prior to drying. In certain embodiments, the composition when dried to provide the vaccine in the solid dosage format retains at least 45% of the potency of the vaccine prior to drying (i.e. at least 45% of the potency of the initial vaccine). In certain embodiments, the composition when dried to provide the vaccine in the solid dosage format retains at least 50% of the potency of the vaccine prior to drying. In certain embodiments, the composition when dried to provide the vaccine in the solid dosage format retains at least 55% of the potency of the vaccine prior to drying. For live vaccines, a recovery efficiency of at least 70% may be preferable.

The appropriate concentrations and combinations of excipients to ensure that the desired potency is retained after drying may be readily determined by those of skill in the art for a particular vaccine based on the teachings provided in the present application and using methods for measuring potency, which will be known to those of skill in the art and examples of which are described below.

Potency can be measured using in vitro or in vivo methods. For in vitro methods, the retention of the antigenic properties of an inactivated vaccine or the antigenic and/or infectious properties of a live vaccine is quantified using common immunochemical and/or virologic analytical techniques. For IPV, potency may be measured in terms of D-antigen activity, which assesses the amount of D-antigen content in the vaccine using an ELISA (enzyme linked immunosorbent assay). Other immunochemical techniques, such as single radial immunodiffusion assay (SRID) can be used to determine the amount of antigenic potency remaining in influenza virus vaccines. Determination of the retention of antigenic potency may also be assessed using common techniques such as SDS PAGE, two-dimensional electrophoresis, western blotting, chromatography (HPLC, UPLC, SEC etc). For live vaccines, quantification of live vaccine may be the preferred option to determine in vitro potency, independently or additionally to other analytical techniques. Live virus can be quantified by several methods to determine the amount of infectious virus. Techniques such as plaque forming assays, focus forming assays, endpoint dilution assays (to determine the 50% tissue culture infective dose; TCID50), flow cytometry or quantitative polymerase chain reaction (Q-PCR) may be used to determine the amount of infectious virus that is present in a cell line that is permissive to infection by the virus. Antigen potency may also be determined by in vivo vaccine potency assays. Here, vaccine is administered to an animal and the induction of an immune response is assessed. For adenovirus, potency may be measured in terms of adenovirus cellular infectivity.

In order to determine the retention of vaccine potency, the potency of the stabilised solid vaccine must be compared directly after drying, in the same assay, to the initial, stock vaccine. The stabilised, solid vaccine must be reconstituted in a suitable buffer for testing in these assays. The vaccine stock can be in the same solution as the reconstituted vaccine and/or can be in a reference buffer. Both the stock vaccine and the stabilised vaccine may be serially diluted or used at a single dilution. Serial dilutions of the vaccine stock can permit the quantification of the formulated vaccines on a dose response curve. Alternatively, a reference vaccine or antigen standard can be used to quantify both the stock and stabilised vaccine. The amount of potency (antigen and/or infectious dose) in the formulated vaccine can then be determined relative to the stock vaccine and the percentage recovery efficiency can be determined.

### Definitions

Unless otherwise defined, all technical and scientific terms used herein have the meaning commonly understood by a person who is skilled in the art in the field of the present invention.

An aqueous soluble polymer may also be referred to as a water soluble polymer and is used herein to describe polymers that dissolve, disperse or swell in water.

The term “polyvinyl alcohol” or “PVA” as used herein is intended to refer to all types of PVA. As such, the PVA may be short chain PVA, long chain PVA, low molecular weight PVA or high molecular weight PVA.

The term “antioxidant” as used herein refers to a substance that inhibits oxidation.

The term “monosaccharide sugar” refers to a sugar that cannot be hydrolysed to give a simpler sugar. The term “disaccharide sugar” refers to a sugar which is formed when two monosaccharides are joined by glycosidic linkage.

The term “polyol sugar” refers a sugar from the class of polyol sugars. This term encompasses sugar alcohols.

As used herein, “stabilising” a vaccine refers to reducing or preventing loss of potency of the vaccine. Preferably, the composition and method of the invention both chemically and physically stabilise vaccines in solid dosage formats. This allows the vaccines to be stored, handled and used at room temperature. “Stabilising” may also be understood as retaining the potency (antigenicity and/or immunogenicity) of a virus in a vaccine. For IPV, potency may be measured by quantitation of D antigen content and recovery efficiency can be measured by comparing values before and after preparing the solid dosage format. Quantitation of D antigen content may be carried out using ELISA.

“Recovery efficiency” as used herein refers to the potency (antigenicity and/or immunogenicity) of the vaccine in the composition of the invention directly after drying expressed as a percentage of the potency of the vaccine of the initial vaccine stock before drying. Recovery efficiency is therefore a measure of the potency of the vaccine remaining following the drying process, i.e. how much viable vaccine is present after drying. Methods for testing potency of the vaccine will be known to persons of skilled in the art. As described above, different methods are suitable for different vaccine types. For example, in the case of IPV, recovery efficiency means the percentage of the D-antigen activity from an initial vaccine stock that was recovered after preparing the solid dosage format. In the case of adenovirus, recovery efficiency means the percentage of infectious adenovirus of the initial vaccine stock liquid formulation that was recovered after preparing the solid dosage format. Potency or recovery efficiency should be compared using the same test method for both the initial vaccine stock and the solid dosage format.

The words “comprises/comprising” and the words “having/including” when used herein with reference to the present invention are used to specify the presence of stated features, integers, steps or components, but do not preclude the presence or addition of one or more other features, integers, steps, components or groups thereof. The term “consists essentially of” as used herein means that no other components that materially affect the stabilising composition are present. This does not rule out the presence of additional minor components.

As used herein, terms such as “a”, “an” and “the” include singular and plural referents unless the context clearly demands otherwise.

It is appreciated that certain features of the invention, which are, for clarity, described in the context of separate embodiments, may also be provided in combination in a single embodiment. Conversely, various features of the invention which are, for brevity, described in the context of a single embodiment, may also be provided separately or in any suitable sub-combination.

### Example 1—Stabilising Compositions for Poliovirus in a Solid Dosage Format

The formulations shown in Tables 1, 2 and 3 were assessed for recovery efficiency. Initial vaccine stock was added to the formulation. This was then dispensed into microneedle moulds, dried and resuspended in buffer. The D-antigen content was analysed using ELISA to measure potency as described below.

D-Antigen ELISA Assay for IPV Stability

Assay Protocol

Day1—Coating:
Dilute 1:1000 serotype specific capture antibody (NIBSC code:13/222) in carbonate coating buffer (storage at 2-8° C.—6.36 g sodium carbonate and 11.72 g sodium hydrogen carbonate made up to 4 l with deionised H₂O).
Add 50 μl to each well of a 96-well ELISA plate. One plate per poliovirus serotype.
Incubate overnight at 2-8° C. in a box with a humidified atmosphere or closed with a plastic foil.

Day2—Development:
Wash each ELISA plate 4× with wash buffer (Dulbecco's 6 Salt PBS containing 2.0% dried milk and 0.5% Tween 20—prepare on day of assay and discard any unused buffer after use). Leave plate containing last wash at room temperature for at least 30 min.
Prepare independent series twofold dilutions of reference and test vaccines in assay diluent (Dulbecco's 6 Salt PBS containing 2.0% dried milk powder—prepare on day of assay and discard any unused buffer after use).
Add 50 μ/well of vaccine or reference dilution. Seal plates and incubate for at least 2 h at 35-38° C. in a box with a humidified atmosphere or closed with a plastic foil.
Wash 3× with wash buffer.
Add 50 μl/well of monoclonal antibody (NIBSC code:520) diluted 1:500 in assay diluent to each well, including blanks. Seal plates and incubate for at least 1 h but not more than 75 min at 37° C.
Wash 3× with wash buffer.
Add 50 μl/well peroxidase conjugated anti-mouse (Sigma A5906) diluted 1:1000 in assay diluent. Seal plates and incubate for at least 1 h but not more than 75 min at 37° C.
Wash 3× with PBS.
Prepare OPD substrate:
   Substrate buffer—Mix 12.15 ml 0.1 M citric acid, 12.85 ml 0.2 M Na 2 HPO 4, and 25 ml distilled H2O. Prepare immediately before use.
   Substrate reagents: 0.1 M citric acid-19.2 g made up to 1 l with distilled H2O or 0.1 M citric acid.H2O—21.0 g made up to 1 l with distilled H2O. Storage—room temperature.
   0.2 M Na2HPO4—28.4 g made up to 1 l with distilled H₂O or 0.2 M Na2HPO4.12 H2O—71.63 g made up to 1 l with distilled H2O. Storage—room temperature.
   OPD substrate: Prepare in a 50 ml centrifuge tube: 1×30 mg o−phenylenediamine dihydrochloride substrate tablet (Sigma-Aldrich)+50 ml substrate buffer+50 μl hydrogen peroxide (30%, Sigma-Aldrich). Use within 1 h of addition of tablet to buffer and add H₂O₂ immediately before use (Store in dark).
Add 50 μl/well of OPD substrate. Leave at room temperature in the dark.
Stop reaction after 30 min by addition of 50 μl/well of 1 M H2SO4.
Read optical density at 492 nm as soon as possible.

Results are shown in Tables 1 and 2. A 70% recovery efficiency, for example, means that 70% of the D-antigen activity from the initial vaccine stock was recovered. Arbitrarily, a recovery efficiency of at least 45% was considered to provide an acceptable degree of stability with a higher recovery efficiency of 50% or more being preferable. Results for Salk type 3 and a drying time of 24 hours are shown in Tables 1 and 2. All reported excipients concentrations refer to the final formulation volume in the presence of the vaccine stock and as such can be considered as final concentrations.

TABLE 1
Formulations using Salk type 3 and a drying time of 24 hours

| | IPV-F1 | IPV-F2 | IPV-F3 | IPV-F4 | IPV-F5 | IPV-F6 | IPV-F7 | IPV-F8 | IPV-F9 |
|---|---|---|---|---|---|---|---|---|---|
| Trehalose (% w/v) | 15 | 15 | 15 | 15 | 15 | 25 | 15 | 15 | 15 |
| Sorbitol (% w/v) | 5 | 5 | 5 | 5 | 0 | 0 | 5 | 5 | 5 |
| Sodium | 3 | 3 | 3 | 3 | 3 | 3 | 3 | 3 | 3 |
| Glutamate (% w/v) | | | | | | | | | |
| MgCl₂ (% w/v) | 3 | 3 | 3 | 3 | 3 | 3 | 3 | 3 | 3 |
| PVA (% w/v) | 5 | 0 | 3 | 0 | 3 | 3 | 5 | 3 | 3 |
| Glutathione (mM) | 20 | 20 | 40 | 40 | 40 | 40 | 40 | | |
| Vitamin C (mM) | | | | | | | | 40 | |
| Cys + Glu + Gly (mM) | | | | | | | | | 40 |
| Cys (mM) | | | | | | | | | |
| Glycine (mM) | | | | | | | | | |
| Glutamic Acid (mM) | | | | | | | | | |
| % of | 46% | 70% | 70% | 64% | 23% | 39% | 46% | 73% | 46% |
| initial/stock | | | | | | | | | |
| IPV recovered | | | | | | | | | |

TABLE 2
Formulations using Salk type 3 and a drying time of 24 hours

| | IPV-F10 | IPV-F11 | IPV-F12 | IPV-F13 | IPV-F14 | IPV-F15 | IPV-F16 | IPV-F17 |
|---|---|---|---|---|---|---|---|---|
| Trehalose (% w/v) | 15 | 15 | 15 | 15 | 15 | 15 | 15 | 15 |
| Sorbitol (% w/v) | 5 | 5 | 5 | 5 | 5 | 5 | 5 | 5 |
| Sodium Glutamate (% w/v) | 3 | 3 | 3 | 3 | 3 | 3 | 3 | 3 |
| MgCl2 (% w/v) | 3 | 3 | 3 | 3 | 3 | 3 | 3 | 3 |
| PVA (% w/v) | 3 | 3 | 3 | 5 | 5 | 5 | 5 | 5 |
| Glutathione (mM) | | | | | | | | |
| Vitamin C (mM) | | | | 40 | | | | |
| Cys + Glu + Gly (mM) | | | | | 40 | | | |
| Cys (mM) | 40 | | | | | 40 | | |
| Glycine (mM) | | 40 | | | | | 40 | |
| Glutamic Acid (mM) | | | 40 | | | | | 40 |
| % of | 51% | 60% | 59% | 46% | 51% | 67% | 66% | 63% |
| initial/stock | | | | | | | | |
| IPV recovered | | | | | | | | |

The results shown in Tables 1 and 2 indicate that an optimal concentration of PVA is less than 3%. An optimal glutathione concentration range is 20-80 mM. Results for vitamin C were equivalent to glutathione. Results for cysteine, glutamic acid, glycine or a combination comprising all three were equivalent. Sorbitol in a range of 3-8% w/v was found to be essential for IPV.

Based on the results observed, the inventors provided a preferred final formulation having the excipients shown in Table 3.

TABLE 3
Final Formulation using Salk type
1, 2 or 3 and a drying time of 36 hours

| | |
|---|---|
| Trehalose (% w/v) | 20 |
| Sorbitol (% w/v) | 3 |
| Sodium Glutamate (% w/v) | 3 |
| MgCl2 (% w/v) | 3 |
| PVA (% w/v) | 1.5 |
| Glutathione (mM) | 40 |
| Recovery efficiency TYPE 1 | 110% |
| Recovery efficiency TYPE 2 | 100% |
| Recovery efficiency TYPE 3 | 85% |

The final formulation as shown in Table 3 provided surprisingly high IPV recovery efficiency values of 110%, 100% and 85% for Salk types 1, 2 and 3 respectively. As such, the composition and method of the invention assist in preserving D-antigen content during formation of the solid dosage format such that there is no loss in the potency of the vaccine.

### Example 2—Microneedles

The final formulation of Table 3 containing Salk IPV type 1-2-3 was incorporated into dissolvable microneedle patches as an example of a potential solid-state vaccine administration platform. These were shown to exhibit strong mechanical robustness, as exemplified by mechanical strength and skin penetration of the dissolvable microneedle patches containing the IPV in the final formulation.

### Example 3—Stabilising Compositions for Adenovirus in a Solid Dosage Format (Films)

The stabilising effect of the composition of the invention for a DNA virus, adenovirus, was also investigated. Formulated adenoviruses were dried as thin layers. This dosage form mimics the production of films and wafers used for oral vaccination, for example.

Adenovirus stability was assessed with formulations described in Table 4 (drying time of 30 hours). The formulations containing luciferase expressing-Adenoviruses (Ad-luc) were dried in thin layers on a PDMS support as an example of a potential solid-state vaccine administration platform, such as oral films. PBS serves as a comparison where the initial vaccine stock was formulated in only PBS rather than in the compositions of the invention.

Materials
Dulbecco's Modified Eagles Media (DMEM)
L-Glutamine.
Penicillin/Streptomycin solution
Fetal Calf Serum (FCS) Heat inactivate to 65 C for 30 min.
Non-essential amino acids (NEAA)
Phosphate buffered saline
Trypsin LE™ Express
Luciferase assay kit,

To Make Up Complete Medium (D-10): (DMEM with 10% FCS):
435 ml DMEM
50 ml FCS
5 ml L-glutamine
5 ml P/S
5 ml NEAA

Luciferase Assay Protocol to Evaluate Ad Stability
1) Plate HEK 293A cells into each well of a 24 well plate (×2).
2) Incubate plates at 37° C., 5% CO₂ for a minimum of 2 hours, this allows cells to adhere to the bottom of the plates
3) Standard curve preparation: Make series of dilutions ( 1/10) of the adenovirus stock at an appropriate starting concentration to provide a linear dilution series.
4) Add 100 μl of each of the std curve dilutions or samples drop wise to each well on the 24 plate. Plates are returned to the incubator for 48 hours to allow the infection to establish.
5) Remove growth medium from cultured cells and rinse cells in 1×PBS. Do not dislodge cells and remove as much of the final wash as possible.
6) Dispense a 120 μl of 1× lysis reagent (CCLR) into each culture vessel and leave it there for 2 minutes.
7) Dislodge the cell layer pipetting up and down into the 24 well plate. Centrifuge the well at 12,000×g for 15 seconds (at room temperature) or up to 2 minutes (at 4° C.) and transfer 20 μl of cell lysate to a new 96 wells plate.
8) Mix 20 μl of cell lysate with 100 μl of Luciferase Assay Reagent and measure the light produced for a period of 10 seconds.

All formulations dissolved in less than five minutes when the media was dropped on the dried layer, driving towards a fast dissolving dosage format.

TABLE 4
Formulations using luciferase expressing-
Adenoviruses (Ad-luc) and a drying time of 30 hours

| | | Adenovirus formulated with: | | |
|---|---|---|---|---|
| | | A | B | C |
| Trehalose (% w.v) | | 20 | 30 | 25 |
| Sorbitol (% w/v) | | 3 | 3 | 3 |
| Sodium Glutamate (% w/v) | | 3 | 0.5 | 0.5 |
| MgCl2 (% w/v) | | 3 | 0.5 | 0.5 |
| PVA (% w/v) | | 1.5 | 0.5 | 3 |
| Glutathione (mM) | | 40 | 80 | 50 |
| PBS (% v) | 100 | | | |
| Drying time (hours) | 30 | 30 | 30 | 30 |
| % of initial Ad-luc | 3 | 76 | 52 | 50 |
| recovery compared with | | | | |
| liquid formulation | | | | |

Notably the formulation of Table 3 (denoted A in Table 4) provided a recovery efficiency of 76% for adenovirus.

### Example 4—Stabilising Effect of Glutathione

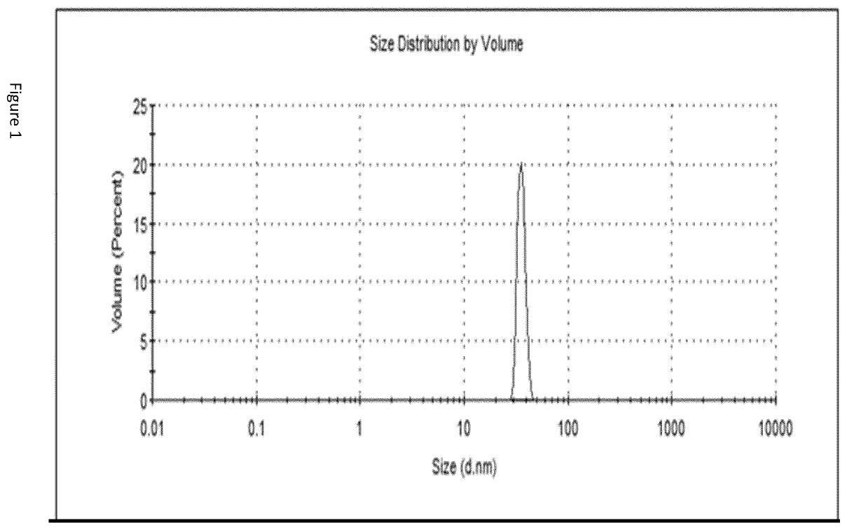

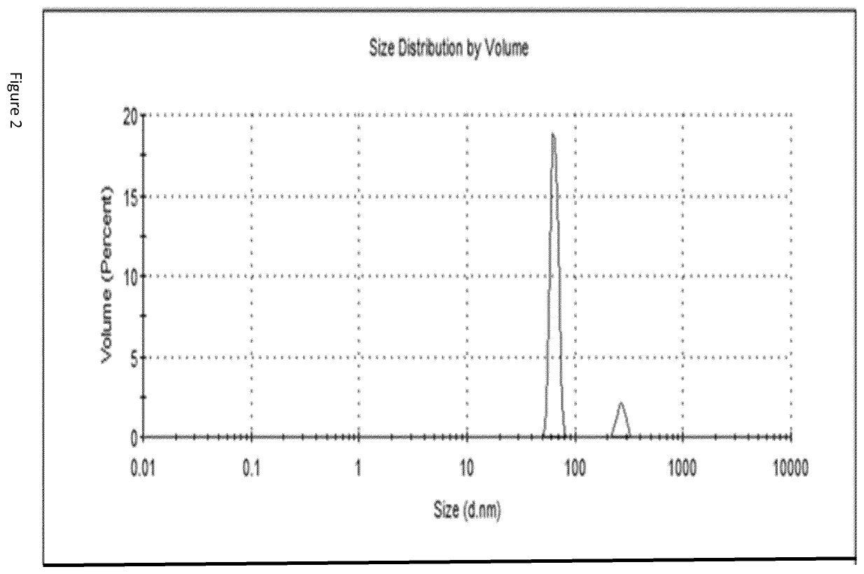

IPV type 3 was tested using dynamic light scattering (DLS) to analyse its particle size after a buffer change process in saline solution, subsequently formulated with water and exposed at +20° C./10 mBar for 24 hours drying. The virus was resuspended in water for the analysis. The size distribution was compared with the results obtained analysing fresh liquid IPV type 3 after a buffer change process in saline solution and stored at +4° C. The results for the fresh liquid are shown in Table 5 and FIG. 1. The results for IPV after drying are shown in Table 5 and FIG. 2.

TABLE 5
Unformulated and stock IPV

| | Fresh liquid | IPV type 3 (after buffer |
|---|---|---|
| | IPV type 3 | change) diluted in water, |
| | (after buffer change) | after drying at |
| Size: | stored at +4° C. | +20° C./10 mBar |
| Z-average Peak 1 | 35.76 (100%) | 64.24 (89.9) |
| (% volume) | | |
| Z-average Peak 2 | 0 | 267.0 (10.1) |
| (% volume) | | |
| PDI | 0.287 | 0.465 |

The results show an increased size of particles (from 35.76 nm to 64.24 nm—FIG. 2) and the presence of a secondary peak in the distribution graph of IPV type 3 after a drying process at 20° C./10 mBar for 24 hours when formulated with de-ionised water compared to the fresh liquid IPV type 3 (FIG. 1). The agglomeration process of virus particles occurs when they are exposed to a higher temperature than the required +4° C., such as +20° C., due to a progressive denaturation and hydrophobic interactions of virus surface proteins.

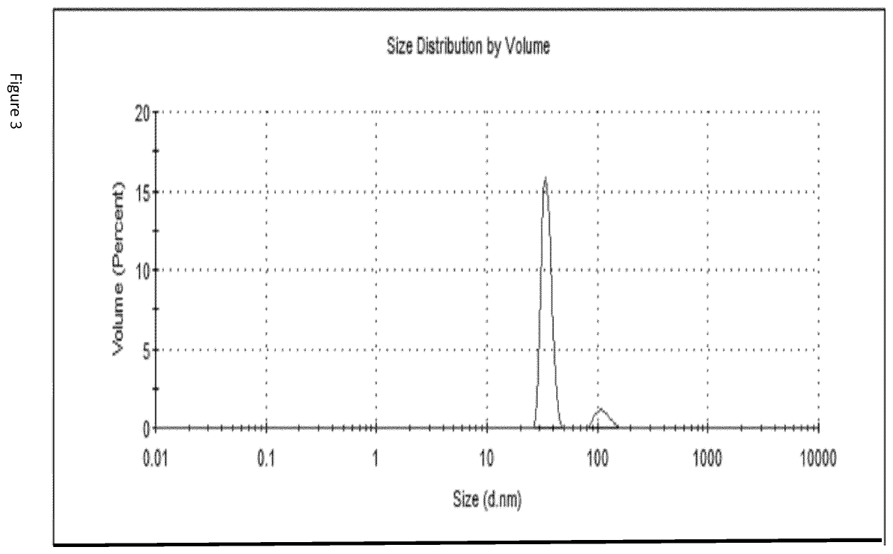

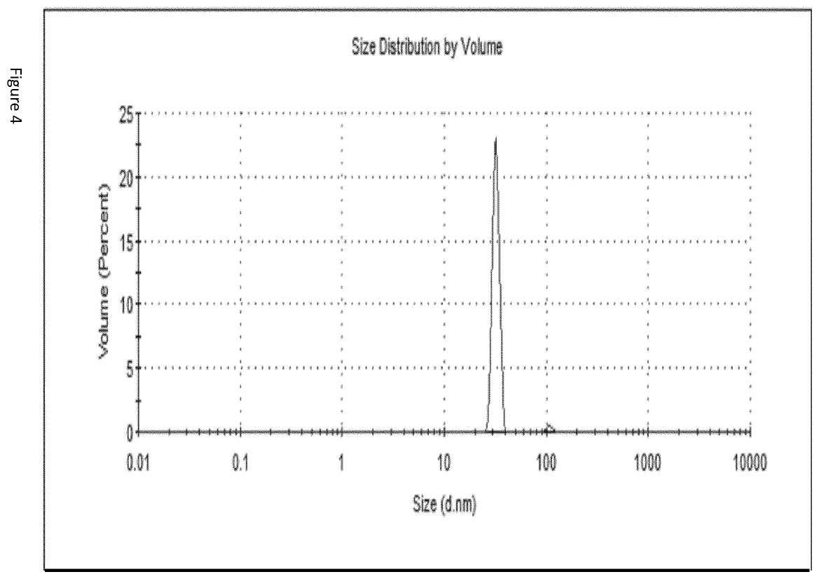

IPV type 3 was formulated in formulations IPV-F18 and IPV-F19 and dried in a tube at +20° C./10 mBar. Results shown in FIGS. 3 and 4 and corresponding Table 6 show that glutathione is able to stabilise IPV type 3 (Salk) during the drying process (27 nm is the IPV virus size when analyzed directly after the buffer change processes reported above). Samples were changed in buffer before being analysed using DLS at 4° C.

TABLE 6
Formulated IPV

| | IPV-F18 | IPV-F19 |
|---|---|---|
| Trehalose (% w/v) | | 15 |
| Sorbitol (% w/v) | | 10 |
| Glutathione (mM) | 20 | 20 |
| Size: | | |
| Z-average Peak 1 (% volume) | 110.6 (8.7%) | 32.18 (98.1%) |
| Z-average Peak 2 (% volume) | 34.75 (91.3%) | 107.6 (1.9%) |
| PDI | 0.331 | 0.238 |

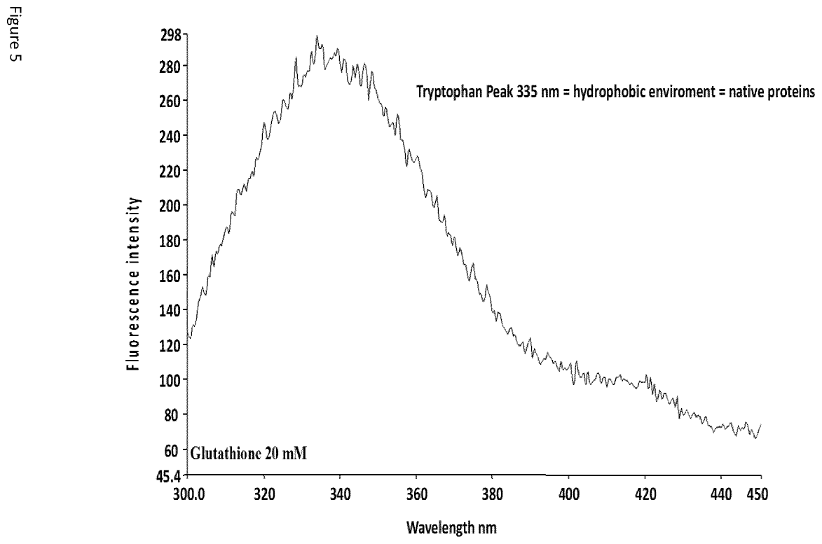

FIG. 5 shows the native conformation of IPV type 3 (Salk) after the drying process at +20° C./10 mBar when formulated as reported in Table 6 above and analysed for its intrinsic fluorescence The intrinsic fluorescence analysis shows the tryptophan exposition in the hydrophobic environment and consequently the native conformation of virus surface proteins.

The observed behaviour confirms the important role of the claimed excipients in vaccine stabilisation during the drying process.

1. A composition for stabilising a vaccine in a solid dosage format, the composition comprising:
an antioxidant;
a monosaccharide or disaccharide sugar;
a polyol sugar;
one or more salts;
a vaccine; and
optionally an aqueous soluble polymer,
wherein the composition is formulated such that at least 45% of the initial potency of the vaccine is retained subsequent to stabilising the vaccine in the solid dosage format.

2. The composition as claimed in claim 1, wherein the composition comprises:
20 to 80 mM antioxidant;
10 to 40% w/v monosaccharide or disaccharide sugar;
3-8% w/v polyol sugar;
0.5-8% w/v of each of the one or more salts; and
0-5% w/v aqueous soluble polymer.

3. The composition as claimed in claim 1 or 2, wherein:
the antioxidant is selected from the group consisting of glutathione, vitamin C, glycine, cysteine, glutamic acid and a combination of glycine, cysteine and glutamic acid;
the monosaccharide or disaccharide sugar is the disaccharide sugar trehalose;
the polyol sugar is sorbitol;
the aqueous soluble polymer is polyvinyl alcohol (PVA); and/or
the one or more salts comprise magnesium chloride and/or sodium glutamate.

4. The composition as claimed in any one of claims 1 to 3, wherein the composition comprises:
20 to 80 mM antioxidant selected from the group consisting of glutathione, vitamin C, glycine, cysteine, glutamic acid and a combination of glycine, cysteine and glutamic acid;
10 to 40% w/v trehalose;
3-8% w/v sorbitol;
0-5% w/v PVA;
0.5-5% w/v magnesium chloride; and
0.5-5% w/v sodium glutamate.

5. The composition as claimed in any one of claims 1 to 4, wherein the composition comprises:
40 mM glutathione;
20% w/v trehalose;
3% w/v sorbitol;
1.5% w/v PVA;
3% w/v magnesium chloride; and
3% w/v sodium glutamate.

6. A composition for stabilising a vaccine in a solid dosage format, the composition comprising:
20 to 80 mM antioxidant;
10 to 40% w/v monosaccharide or disaccharide sugar;
3-8% w/v polyol sugar;
0.5-8% w/v of each of one or more salts;
0-5% w/v aqueous soluble polymer; and
a vaccine.

7. The composition as claimed in claim 6, wherein:
the antioxidant is selected from the group consisting of glutathione, vitamin C, glycine, cysteine, glutamic acid and a combination of glycine, cysteine and glutamic acid;
the monosaccharide or disaccharide sugar is the disaccharide sugar trehalose;
the polyol sugar is sorbitol;
the aqueous soluble polymer is polyvinyl alcohol (PVA); and/or
the one or more salts comprise magnesium chloride and sodium glutamate.

8. The composition as claimed in claim 7, wherein the composition comprises:
20 to 80 mM antioxidant selected from the group consisting of glutathione, vitamin C, glycine, cysteine, glutamic acid and a combination of glycine, cysteine and glutamic acid;
10 to 40% w/v trehalose;
3-8% w/v sorbitol;
0-5% w/v PVA;
0.5-5% w/v magnesium chloride; and
0.5-5% w/v sodium glutamate.

9. The composition as claimed in claim 8, wherein the composition comprises:
20 to 80 mM antioxidant selected from the group consisting of glutathione, vitamin C, glycine, cysteine, glutamic acid and a combination of glycine, cysteine and glutamic acid;
15 to 30% w/v trehalose;
3-5% w/v sorbitol;
0-5% w/v PVA;
0.5-3% w/v magnesium chloride; and
0.5-3% w/v sodium glutamate.

10. The composition as claimed in claim 9, wherein the composition comprises:
20 to 40 mM antioxidant selected from the group consisting of glutathione, vitamin C, glycine, cysteine, glutamic acid and a combination of glycine, cysteine and glutamic acid;
15 to 25% w/v trehalose;
5% w/v sorbitol;
0-5% w/v PVA;
3% w/v magnesium chloride; and
3% w/v sodium glutamate.

11. The composition as claimed in claim 10, wherein the composition comprises 40 mM antioxidant.

12. The composition as claimed in claim 9, wherein the composition comprises:
40 to 80 mM antioxidant selected from the group consisting of glutathione, vitamin C, glycine, cysteine, glutamic acid and a combination of glycine, cysteine and glutamic acid;
20 to 30% w/v trehalose;
3% w/v sorbitol;
0.5-3% w/v PVA;
0.5-3% w/v magnesium chloride; and
0.5-3% w/v sodium glutamate.

13. The composition as claimed in claim 12, wherein the antioxidant is glutathione.

14. The composition as claimed in claim 13, wherein the composition comprises:
40 mM glutathione;
20% w/v trehalose;
3% w/v sorbitol;
1.5% w/v PVA;
3% w/v magnesium chloride; and
3% w/v sodium glutamate.

15. The composition as claimed in any one of claims 1 to 14, wherein the composition is formulated such that at least 50% of the initial potency of the vaccine is retained subsequent to stabilising the vaccine in the solid dosage format.

16. The composition as claimed in any one of claims 1 to 15, wherein the composition is dried to provide the vaccine in the solid dosage format.

17. A method of stabilising a vaccine in a solid dosage format, the method comprising the steps of:
providing a composition as claimed in any one of claims 1 to 15; and
drying the composition to provide the vaccine in the solid dosage format.

18. The method as claimed in claim 17, wherein providing a composition comprises providing a composition as claimed in claim 5.

19. The method as claimed in claim 17, wherein providing a composition comprises providing a composition as claimed in claim 14.

20. The method as claimed in any one of claims 17 to 19, wherein drying the composition to provide the vaccine in the solid dosage format does not comprise lyophilisation.

21. The method as claimed in any one of claims 17 to 20, wherein drying is carried out at ambient temperature.

22. Use of a composition according to any one of claims 1 to 15 for stabilising a vaccine in a solid dosage format.

23. The composition as claimed in any one of claims 1 to 16, the method as claimed in any one of claims 17 to 21, or the use as claimed in claim 22, wherein the solid dosage format is selected from the group consisting of microneedles, capsules, wafers, microneedle patches and patches.

24. The composition, method or use as claimed in claim 23, wherein the solid dosage format is microneedle patches.

25. The composition, method or use as claimed in claim 23, wherein the solid dosage format is wafers.

26. The composition as claimed in any one of claims 1 to 16, the method as claimed in any one of claims 17 to 21, or the use as claimed in claim 22, wherein the solid dosage format is formulated for administration orally.

27. The composition as claimed in any one of claims 1 to 16, the method as claimed in any one of claims 17 to 21, or the use as claimed in claim 22, wherein the solid dosage format is formulated for transcutaneous administration.

28. The composition as claimed in any one of claims 1 to 16 and 23 to 27, the method as claimed in any one of claims 17 to 21 and 23 to 27, or the use as claimed in any one of claims 22 to 27, wherein the vaccine comprises an RNA virus.

29. The composition, method or use as claimed in claim 28, wherein the RNA virus comprises a picornavirus.

30. The composition, method or use as claimed in claim 29, wherein the picornavirus is poliovirus.

31. The composition, method or use as claimed in claim 30, wherein the poliovirus is Salk Inactivated Polio Vaccine (IPV) or Sabin oral or inactivated poliovirus vaccine.

32. The composition, method or use as claimed in claim 30, wherein the composition is the composition as claimed in claim 10 or 11.

33. The composition as claimed in any one of claims 1 to 16 and 23 to 27, the method as claimed in any one of claims 17 to 21 and 23 to 27, or the use as claimed in any one of claims 22 to 27, wherein the vaccine comprises a DNA virus.

34. The composition, method or use as claimed in claim 33, wherein the DNA virus comprises adenovirus.

35. The composition, method or use as claimed in claim 34, wherein the composition is the composition as claimed in claim 12 or 13.
